(19) United States
(12) Patent Application Publication
(10) Pub. No.: US 2004/0167064 A1
(43) Pub. Date: Aug. 26, 2004

(54) Materials and methods relating for the treatment and diagnosis of pre-eclampsia

(76) Inventors: Julian Schofield, London (UA); Thomas William Rademacher, Oxford (UA); Sylvie Deborde, St. Michel Mt. Mercure (FR)

Correspondence Address: QUINE INTELLECTUAL PROPERTY LAW GROUP, P.C., P O BOX 458, ALAMEDA, CA, 94501, US

(21) Appl. No.: 10/311,387

(22) PCT Filed: Jun. 25, 2001
(86) PCT No.: PCT/GB01/02800

(30) Foreign Application Priority Data
Jun. 26, 2000 (GB) 0015625.7

(51) Int. Cl. A61K 38/17

(52) U.S. Cl. 514/12

(57) ABSTRACT

The present invention relates to use of GPI-PLD antagonists for the prevention, treatment and diagnosis of pre-eclampsia. The substantial GPI-PLD activity is present in the placenta in pre-eclampsia is not expressed in the placenta, but rather is taken up from the material circulation. As a result, abnormal or dysregulated GPI-PLD activity present in the placenta in pre-eclampsia may be correctable by administration of GPI-PLD to the mother to correct the problems caused by abnormal or dysregulated GPI-PLD, e.g. to reduce the abnormal release and in situ production of placental IPGs involved in the pathogenesis of pre-eclampsia. This can be achieved using exogenous GPI-PLD or a fragment thereof, e.g. an inactive GPI-PLD capable of competing with or displacing the abnormal or dysregulated GPI-PLD, e.g. from Apo-A1.

### FIELD OF THE INVENTION

The present invention relates to materials and methods for the treatment and diagnosis of pre-eclampsia, and in particular to the use of glycosylphosphatidylinositol specific phospholipase D (GPI-PLD) proteins.

### BACKGROUND OF THE INVENTION

Pre-eclampsia is a placental disease (Redman, 1991) characterised by insufficiency of the uteroplacental circulation (Robertson et al, 1967). It is a potentially fatal condition, affecting about 10% of all pregnancies, and is a major factor in the perinatal mortality rate. There is evidence that one or more placentally-derived factors are released into the maternal circulation which either directly or indirectly cause maternal endothelial dysfunction and ensuing maternal problems with activation of the clotting system, increased vascular permeability and ischaemia in maternal organs secondary to vasoconstriction (Roberts et al, 1989). A further notable feature of the disease is a large abnormal increase in glycogen accumulation in the placenta, suggesting dysregulated carbohydrate metabolism (Arkwright et al, 1993).

Inositol phosphoglycans (IPGs) have been implicated as mediators in the signalling pathways activated by insulin binding to its receptor. Two different classes of IPG activity have been identified, designated A and P-type. Both classes are thought to be generated by phospholipase action on membrane glycosylphosphatidylinositol (GPI). Previous investigations have shown an extremely high level of P-type IPGs in the placenta, with greater than two-fold more P-type IPGs in a pre-eclamptic placenta at term than a normal placenta. IPGs are also increased in the urine of pre-eclamptic patients, and the level of IPGs is correlated with other markers of pre-eclampsia such as proteinuria and plasma alanine-aspartate transaminase activity (see WO98/10791). It is therefore possible that abnormal placental carbohydrate metabolism in pre-eclampsia is a consequence of the elevated levels of IPGs. However, the cause of the high IPG levels has not been determined.

GPI-PLD proteins are abundant in bovine and human serum. It is thought that they are primarily synthesised by the Langerhans cells of the pancreas and released into the circulation in a complex with apolipoprotein A1 (ApoA1) (Hoener et al 1993). The GPI-PLD enzyme is capable of hydrolysing glycosylphosphatidylinositol, by which a number of cell surface proteins are attached to cell membranes. GPI-PLD cleaves a phosphodiester bond, to liberate phosphatidic acid, in contrast to phospholipase C activity, which generates diacyl glycerol.

A further proposed function of GPI-PLD is to produce inositolphosphoglycans by cleavage of “free” GPIs in the plasma membrane in response to a growth factor or hormone binding to its receptor (Rademacher et al, 1994). This role for GPI-PLD has been demonstrated in mast cells where IgE-dependent activation of these cells results in release of their granule contents, which include substances such as histamine, a mediator of the inflammatory response. In the presence of antigen, histamine is released; this release can be mimicked by addition of IPGs and is blocked by addition of anti-GPI-PLD antibodies (Lin et al, 1991).

U.S. Pat. No. 5,418,147 (Huang et al) describes the purification of GPI-PLD from bovine liver, and the subsequent cloning of three GPI-PLD enzymes from bovine liver, human liver and human pancreas cDNA libraries. This patent reports the full length cDNA and amino acid sequences of the GPI-PLDs from human and bovine liver, and the partial cDNA and amino acid sequences of the human pancreatic form of the enzyme. Subsequently, the full length sequence of the pancreatic form of GPI-PLD was reported in Tsang et al (1992), and this enzyme has been found in cDNA libraries from breast, eye, spleen and tonsil. The three forms of the enzymes are highly homologous with the predicted mature protein sequences of bovine liver GPI-PLD sharing 82% sequence identity with the human liver enzyme and 81% sequence identity with the human pancreatic enzyme. The amino acid sequences of human liver and pancreatic forms of GPI-PLD were deposited at GenBank under accession numbers L11701 and L11702 and consist of 841 and 840 amino acids respectively. The human liver and pancreatic forms of GPI-PLD share 94.6% sequence identity. The structure of GPI-PLDs is further discussed in Scallon et al, 1991.

### SUMMARY OF THE INVENTION

Broadly, the present invention relates to use of GPI-PLDs for the prevention, treatment and diagnosis of pre-eclampsia. Without wishing to be bound by any particular theory, the present invention is based on the realisation that a substantial GPI-PLD activity is present in the placenta, but that the protein responsible for this enzymatic activity may not itself be expressed in the placenta. Rather, it may be taken up into the placenta from the maternal circulation. As a result, abnormal or dysregulated GPI-PLD activity present in the placenta in pre-eclampsia may be correctable by administration of GPI-PLD to the mother, in such a way that the properties of the circulating GPI-PLD are altered. This may be treatable by administration of GPI-PLD to correct the problems caused by abnormal or dysregulated GPI-PLD, e.g. to reduce the abnormal release and in situ production of placental IPGs involved in the pathogenesis of pre-eclampsia. In an alternative embodiment, the present invention provides for the administration of a GPI-PLD antagonist, e.g. an inactive GPI-PLD capable of competing with or displacing the abnormal or dysregulated GPI-PLD, e.g. from Apo-A1.

Accordingly, in a first aspect, the present invention provides the use of an antagonist of endogenous glycosylphosphatidylinositol phospholipase D (GPI-PLD) for the preparation of a medicament for the prevention or treatment of pre-eclampsia.

In one embodiment, the GPI-PLD antagonist competes with or displaces endogenous GPI-PLD which causes pre-eclampsia. The competition may take place in the circulation, thereby reducing the amount of endogenous GPI-PLD reaching and being taken up by the placenta. The competition may displace and/or replace endogenous GPI-PLD which is bound to a carrier such as apolipoprotein A1. Alternatively or additionally, the exogenous GPI-PLD may be taken up by the placenta and compete there with the endogenous enzyme, thereby ameliorating its detrimental effect in causing pre-eclampsia.

The GPI-PLD antagonist may be a GPI-PLD protein or a fragment thereof. In one embodiment, the GPI-PLD is inactive or has a reduced activity, e.g. the activity of cleaving of a phosphodiester bond of a glycosylphosphatidylinositol. In the context of the treatment of pre-eclampsia, such GPI-PLD variants can reduce the production of P-type inositol phosphoglycans, elevated levels of which are known to be present in pre-eclamptic placenta and are associated with this condition.

In a further aspect, the present invention provides the use of a nucleic acid molecule encoding GPI-PLD for the preparation of a medicament for the prevention or treatment of pre-eclampsia.

In a further aspect, the present invention provides the use of a host cell capable of expressing and secreting GPI-PLD in the preparation of a medicament for the prevention or treatment of pre-eclampsia. In one embodiment, the host cell is encapsulated, e.g. in a biocompatible polymer, so that the GPI-PLD produced by the host cell can be secreted into the patient, while preventing rejection of the host cell by the immune system of the patient. Methods for encapsulating cells in biocompatible polymers are described in WO93/16687 and WO96/31199.

In a further aspect, the present invention provides a kit comprising a composition including GPI-PLD for the prevention or treatment of pre-eclampsia. The composition comprising GPI-PLD may be administered alone, or in conjunction with other medicaments for the treatment of pre-eclampsia, for either simultaneous or sequential administration.

In a further aspect, the present invention provides a method of diagnosing a patient who has or at risk of developing a pre-eclampsia, the method comprising determining the amount of GPI-PLD and/or GPI-PLD activity in a sample obtained from the patient.

In one embodiment, the present invention provides a method of diagnosing a patient who has or at risk of developing pre-eclampsia, the method comprising the steps of:

(a) contacting a sample obtained from the patient with a solid support having immobilised thereon a binding agent having binding sites specific for GPI-PLD;

(b) determining the amount of GPI-PLD or the activity of GPI-PLD which binds to the binding agent.

The method may involve the additional step of:

(c) correlating the value obtained in step (b) with measurements obtained from control subjects to determine whether the patient has or is at risk of developing pre-eclampsia.

These and other aspects of the present invention are described in more detail below. By way of example and not limitation, embodiments of the present invention will be described with reference to the accompanying figures.

### BRIEF DESCRIPTION OF THE FIGURES

FIG. 1 shows a Western blot showing the presence of GPI-PLD in homogenate of placenta (lane 1) and microvilli preparations of normal (lanes 2, 3, 4, 6) and pre-eclamptic (lane 5) placenta.

FIGS. 2 and 3 provide confirmation of the presence of a GPI-specific phospholipase activity in placental microvilli using hydrolysis of mfVSG.

FIG. 4 shows an autoradiograph of a TLC plate showing that the GPI-specific phospholipase activity present in placental microvilli is of the phospholipase D type.

FIG. 5 show the results of RT-PCR (reverse transcriptase polymerase chain reaction) reactions showing that the gene encoding GPI-PLD is not expressed in either normal or pre-eclamptic placentae.

FIG. 6 shows an alignment of the deduced amino acid sequences of human liver GPI-PLD disclosed in PCT/GB99/04399 the bovine and human liver GPI-PLD sequences disclosed in U.S. Pat. No. 5,418,147 (Huang et al).

### DETAILED DESCRIPTION

GPI-PLD Proteins

The term “GPI-PLD biological activity” is herein defined as the enzymatic activity of GPI-PLD in liberating phosphatidic acid from a glycosylphosphatidylinositol by cleavage of a phosphodiester bond, e.g. releasing a GPI-anchored protein or generating an inositolphosphoglycan (IPG) from a membrane bound glycosylphosphatidylinositol (GPI). As noted in Heller et al (1994), this activity has been localised to the N-terminal 39 kD portion of full length GPI-PLD.

The present invention can be carried out using a number of classes of substances to ameliorate the effects of endogenous GPI-PLD in placenta, e.g. the dysregulation resulting from endogenous, maternal GPI-PLD being taken up by the placenta.

Firstly, exogenous GPI-PLD can be administered to a patient to compete with the endogenous enzyme, e.g. displacing it from apolipoprotein A1, a GPI-PLD carrier, thereby reducing the amount of the endogenous enzyme reaching the placenta and causing the dysregulation. The exogenous GPI-PLD can further compete with the endogenous enzyme in the placenta, ameliorating the detrimental effects of the endogenous enzyme. In this latter embodiment, the GPI-PLD may be administered with a carrier, and in particular ApoA1, to provide an alternative circulating source of the enzyme.

A further class of GPI-PLD substances are GPI-PLD substances or fragments which are enzymatically inactive or have a reduced biological activity. These substances act as competitive antagonists, either competing with endogenous enzyme for uptake by the placenta, e.g. by displacing endogenous GPI-PLD from ApoA1, or by competing with endogenous enzyme in the placenta thereby to reduce one or more of the detrimental effects of the endogenosu enzyme.

Based on the above considerations, the medical uses of GPI-PLD described herein can employ the GPI-PLD variants disclosed by Huang et al, and in PCT/GB99/04399. The skilled person can use the techniques described herein and others well known in the art to produce large amounts of these proteins, or fragments, active portions, variant and alleles thereof, for use as pharmaceuticals.

It is preferred that GPI-PLD variants for use in the present invention retain the enzymatic activity of GPI-PLD in liberating phosphatidic acid from a glycosylphosphatidylinositol by cleavage of a phosphodiester bond.

In other embodiments, the GPI-PLD may be a non-enzymatically active variant, or a variant having reduced GPI-PLD activity, that may be used to treat pre-eclampsia where the over activity of abnormal or dysregulated GPI-PLD is a causative factor of this condition. In this case, the non-enzymatically active variants can compete with or displace the abnormal or dysregulated GPI-PLD, e.g. from its Apo-A1 carrier, thereby having the effect of down regulating the GPI-PLD activity present in the placenta. The design and synthesis of such competitive antagonists can be readily carried out using the information about the GPI-PLD provided in this application.

GPI-PLD proteins which are amino acid sequence variants or alleles of these prior art sequences can also be used in the present invention. A polypeptide which is a variant or allele may have an amino acid sequence which differs from that given by one or more of addition, substitution, deletion and insertion of one or more amino acids. In some embodiments, the polypeptides have GPI-PLD enzymatic function as defined above. In other embodiments, the GPI-PLDs are enzymatically inactive, e.g. not having the activity of releasing IPGs, and can compete with the abnormal or dysregulated GPI-PLD to down regulate the abnormal GPI-PLD activity in the placenta.

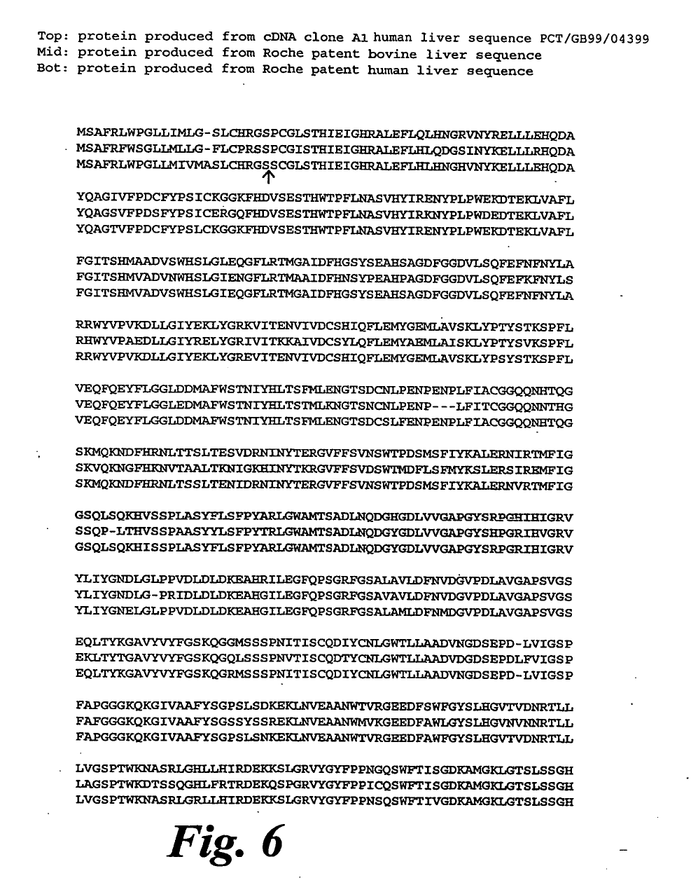

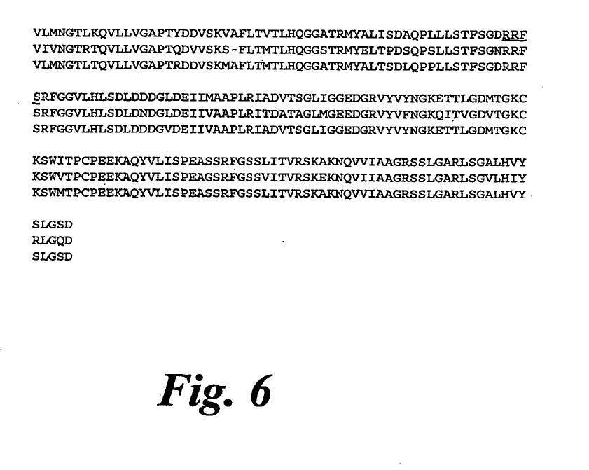

A GPI-PLD protein which is an amino acid sequence variant or allele of an amino acid sequence shown in FIG. 6 may comprise an amino acid sequence which shares greater than about 70%, greater than about 80%, greater than about 90%, greater than about 95%, greater than about 97%, greater than about 98% or greater than about 99% sequence identity with an amino acid sequence shown in FIG. 6. Sequence comparison and identity calculations may be carried out using the Cluster program (Thompson et al, 1994), using the following parameters (Pairwise Alignment Parameters: Weight Matrix: pam series; Gap Open Penalty: 10.00; Gap Extension Penalty: 0.10). Alternatively, the GCG program could be used which is available from Genetics Computer Group, Oxford Molecular Group, Madison, Wis., USA, Version 9.1. Particular amino acid sequence variants may differ from those shown in FIG. 6 by insertion, addition, substitution or deletion of 1 amino acid, 2, 3; 4, 5-10, 10-20 20-30, 30-50, 50-100, 100-150, or more than 150 amino acids.

In one embodiment, the variant GPI-PLD polypeptides of the present invention differ in amino acid sequence as compared to human GPI-PLD at the phosphorylation site from amino acids 689 to 692 of the mature sequence, i.e. within the amino acid motif RRFS. The term “variant GPI-PLD polypeptide” is intended, inter alia, to include polypeptides which are modified within this region by deletion, substitution and/or insertion of one or more amino acids. These sequence differences may be the result of varying the GPI-PLD amino acid sequence of a parent GPI-PLD polypeptide, either a wild type GPI-PLD polypeptide or a GPI-PLD polypeptide comprising one or more other modifications, e.g. by manipulation of the nucleic acid encoding the polypeptide, by altering the polypeptide itself or by the de novo synthesis of the variant protein. In preferred embodiments, the GPI-PLD retains, at least in part, one of its biological activities, e.g. by including a functional N-terminal domain.

A deletion may take the form of the deletion of one, two, three or all four amino acids within this region. In some embodiments, the deletion may be part of a larger deletion encompassing a greater part of the GPI-PLD molecule. In a preferred embodiment, the variant GPI-PLD polpeptides have an amino acid sequence which differs from the amino acid sequence of human wild type GPI-PLD by the deletion comprising residues 689 to 692 inclusive. An insertion may take the form of 1, 2, 3, 4 or 5 or more additional amino acids inserted between amino acids within the RRFS motif to disrupt it.

A substitution may take the form of the substitution of one, two, three or all of the four amino acids within the region corresponding to amino acids 689 to 692 of wild type human GPI-PLD. The substitutions within this region may be part of a more extensive series of substitutions encompassing other parts of the GPI-PLD polypeptide. In particular, mutant forms of GPI-PLD which may-have practical use differ from the wild type sequence. Some of these mutants are used in the experiments described below.

These sequence differences may be the result of varying the GPI-PLD amino acid sequence of a parent GPI-PLD polypeptide, either a wild type GPI-PLD polypeptide or a GPI-PLD polypeptide comprising one or more other modifications, e.g. by manipulation of the nucleic acid encoding the polypeptide, by altering the polypeptide itself or by the de novo synthesis of the variant protein. In preferred embodiments, the GPI-PLD retains, at least in part, one of its biological activities, e.g. by the presence of a functional N-terminal domain.

The present invention also includes the use of active portions and fragments of the GPI-PLD proteins for the preparation of a medicament for the prevention or treatment of pre-eclampsia.

An “active portion” of GPI-PLD protein is a polypeptide which is less than said full length GPI-PLD protein, but which retains at least its essential enzymatic activity, e.g. the enzyme activity mentioned above known to be located in the N-terminal 39 kD portion of the enzyme. For instance, portions of GPI-PLD protein can act as sequestrators or competitive antagonists by interacting with other proteins. Typically, active portions comprise at least about 300 amino acids of GPI-PLD, more preferably at least 500 amino acids, and still more preervably at least 700 amino acids, with, for example, the amino acids being selected from one or more motifs from a sequence shown in FIG. 6.

A “fragment” of the GPI-PLD protein means a stretch of amino acid residues of at least about 5 to 7 contiguous amino acids, often at least about 7 to 9 contiguous amino acids, typically at least about 9 to 13 contiguous amino acids, more preferably at least about 20 to 30 or more contiguous amino acids, more preferably greater than 40 amino acids, more preferably greater than 100 amino acids.

A polypeptide for use in the preparation of a medicament for the prevention or treatment of pre-eclampsia according to the present invention may be isolated and/or purified (e.g. using an antibody) for instance after production by expression from encoding nucleic acid (for which see below). Polypeptides according to the present invention may also be generated wholly or partly by chemical synthesis.

The GPI-PLD polypeptides can also be linked to a coupling partner, e.g. an effector molecule, a label, a drug, a toxin and/or a carrier or transport molecule. Techniques for coupling the peptides of the invention to both peptidyl and non-peptidyl coupling partners are well known in the art. In one embodiment, the carrier molecule is a 16 aa peptide sequence derived from the homeodomain of Antennapedia (e.g. as sold under the name “Penetratin”), which can be coupled to a peptide via a terminal Cys residue. The “Penetratin” molecule and its properties are described in WO91/18981.

GPI-PLD Nucleic Acid

GPI-PLD nucleic acids includes a nucleic acid molecule which has a nucleotide sequence encoding a polypeptide with GPI-PLD activity. Suitable full-length sequences of human and bovine GPI-PLD enzymes have been provided, for example, by Huang et al, Tsang et al (1992) and in PCT/GB99/04399. These forms of GPI-PLD have been mapped to human chromosome 6 and are contained in the 4 centimorgan region of D6S1660-D6S1558 at positions 95.95 and 99.71 (NCBI GeneMap'98). The gene starts in the cytogenic region corresponding to 6p22.3 and extends into 6p21.3. This region also contains the IDDM1 and HLA loci (although the HLA genes map to the adjacent D6S1558-D6S1616 interval). The mouse GPI-PLD gene has also been mapped to chromosome 13, near the fim 1 locus, which is found in humans on chromosome 6.

The GPI-PLD coding sequence may be one of the published sequences referred to above, or a complementary nucleic acid sequence, or it may be a sequence variant, derivative or allele of one of these sequences. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

The encoded polypeptide may comprise an amino acid sequence which differs by one or more amino acid residues from one of the published GPI-PLD amino acid sequences. Use of nucleic acid encoding a polypeptide which is an amino acid sequence mutant, variant or allele of a known GPI-PLD is further envisaged by the present invention. Such polypeptides are discussed below. Nucleic acid encoding such a polypeptide may show greater than about 70% identity, greater than about 80% identity, greater than about 90% identity, greater than about 95% identity, greater than about 98% identity, or greater than about 99% identity with a published coding sequence for a GPI-PLD protein as described above.

The present invention also embraces use of fragments of GPI-PLD nucleic acid sequences, the fragments preferably being at least 12, 15, 30, 45, 60, 120 or 240 nucleotides in length.

Generally, nucleic acid for use according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding all or part of the GPI-PLD gene and/or its regulatory elements can be readily prepared by the skilled person using the information and references contained herein and techniques known in the art. For example, see Sambrook et al, 1989, and Ausubel et al, 1992. These techniques include (i) the use of the polymerase chain reaction (PCR) to amplify samples of such nucleic acid, e.g. from genomic sources, (ii) chemical synthesis, or (iii) amplification in E. coli. Modifications to the GPI-PLD sequences can be made, e.g. using site directed mutagenesis, to provide expression of modified GPI-PLD protein or to take account of codon preference in the host cells used to express the nucleic acid.

In order to obtain expression of the GPI-PLD nucleic acid sequences, the sequences can be incorporated in a vector having control sequences operably linked to the GPI-PLD nucleic acid to control its expression. The use of expression systems has reached an advanced degree of sophistication. The vectors may include other sequences such as promoters or enhancers to drive the expression of the inserted nucleic acid, nucleic acid sequences so that the GPI-PLD protein is produced as a fusion and/or nucleic acid encoding secretion signals so that the polypeptide produced in the host cell is secreted from the cell. GPI-PLD protein can then be obtained by transforming the vectors into host cells in which the vector is functional, culturing the host cells so that the GPI-PLD protein is produced and recovering the GPI-PLD protein from the host cells or the surrounding medium. Prokaryotic and eukaryotic cells are used for this purpose in the art, including strains of E. coli, yeast, and eukaryotic cells such as COS or CHO cells. The choice of host cell can be used to control the properties of the GPI-PLD protein expressed in those cells, e.g. controlling where the polypeptide is deposited in the host cells or affecting properties such as its glycosylation and phosphorylation.

PCR techniques for the amplification of nucleic acid are described in U.S. Pat. No. 4,683,195. In general, such techniques require that sequence information from the ends of the target sequence is known to allow suitable forward and reverse oligonucleotide primers to be designed to be identical or similar to the polynucleotide sequence that is the target for the amplification. PCR comprises steps of denaturation of template nucleic acid (if double-stranded), annealing of primer to target, and polymerisation. The nucleic acid probed or used as template in the amplification reaction may be genomic DNA, cDNA or RNA. PCR can be used to amplify specific sequences from genomic DNA, specific RNA sequences and cDNA transcribed from mRNA, bacteriophage or plasmid sequences. The published GPI-PLD protein nucleic acid sequences readily allow the skilled person to design PCR primers. References for the general use of PCR techniques include Mullis et al, Cold Spring Harbor Symp. Quant. Biol., 51:263, 1987; Ehrlich (ed), PCR Technology, Stockton Press, NY, 1989; Ehrlich et al, Science, 252:1643-1650, 1991; “PCR protocols; A Guide to Methods and Applications”, Eds. Innis et al, Academic Press, New York, 1990.

Nucleic acid for use according to the present invention is obtainable using one or more oligonucleotide probes or primers designed to hybridize with one or more fragments of the nucleic acid sequence shown in the figures, particularly fragments of relatively rare sequence, based on codon usage or statistical analysis. A primer designed to hybridize with a fragment of the nucleic acid sequence shown in the above figures may be used in conjunction with one or more oligonucleotides designed to hybridize to a sequence in a cloning vector within which target nucleic acid has been cloned, or in so-called “RACE” (rapid amplification of cDNA ends) in which cDNA's in a library are ligated to an oligonucleotide linker and PCR is performed using a primer which hybridizes with a GPI-PLD nucleic acid sequence shown in figures and a primer which hybridizes to the oligonucleotide linker.

Such oligonucleotide probes or primers, as well as the full-length sequence (and mutants, alleles, variants and derivatives) are also useful in screening a test sample containing nucleic acid for the presence of alleles, mutants and variants of GPI-PLD protein, the probes hybridizing with a target sequence from a sample obtained from the individual being tested. The conditions of the hybridization can be controlled to minimise non-specific binding, and preferably stringent to moderately stringent hybridization conditions are preferred. The skilled person is readily able to design such probes, label them and devise suitable conditions for the hybridization reactions, assisted by textbooks such as Sambrook et al (1989) and Ausubel et al (1992).

Examples of “stringent conditions” are those which: (1) employ low ionic strength and high temperature for washing, for example 0.015 M sodium chloride/0.0015 M sodium citrate/0.1% sodium dodecyl sulphate at 50° C.; (2) employ during hybridisation a denaturing agent, such as formamide, for example, 50% (v/v) formamide with 0.1% BSA/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulphate at 42° C., with washes at 42° C. in 0.2×SSC and 50% formamide at 55° C., followed by high stringency wash consisting of 0.1×SSC containing EDTA at 55° C. These hybridisation conditions may be used in the context of defining nucleic acid sequences which hybridize with GPI-PLD nucleic acid sequences and therefore form part of the present invention.

Uses of GPI-PLD Nucleic Acid

The GPI-PLD nucleic acid sequences can be used in the preparation of cell lines capable of expressing GPI-PLD. Thus, the present invention provides use of a host cell for the preparation of a medicament for the prevention or treatment of pre-eclampsia, e.g. for transplantation into a patient, the host cell being transformed with nucleic acid encoding GPI-PLD, and being capable of expressing and secreting GPI-PLD. In one embodiment, the host cells are encapsulated, e.g. in a biocompatible polymer, so that the GPI-PLD produced by the host cell can be secreted into the patient, while preventing rejection of the host cell by the immune system of the patient. Methods for encapsulating cells in biocompatible polymers are described in WO93/16687 and WO96/31199.

Vectors such as viral vectors have been used in the prior art to introduce genes into a wide variety of different target cells. Typically, the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors other known methods of introducing nucleic acid into cell's includes electroporation, calcium phosphate co-precipitation, mechanical techniques such as microinjection, transfer mediated by liposomes and direct DNA uptake and receptor-mediated DNA transfer.

Drug Formulation

The GPI-PLD enzymes of the invention may be derivatised in various ways. As used herein “derivatives” of the enzymes include salts, coordination complexes with metal ions such as Mn²⁺ and Zn²⁺, esters such as in vivo hydrolysable esters, free acids or bases, hydrates, prodrugs or lipids, coupling partners.

Salts of the enzymes of the invention are preferably physiologically well tolerated and non toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art.

Derivatives which as prodrugs of the enzymes are convertible in vivo or in vitro into one of the active enzymes and activators. Typically, at least one of the biological activities of compound will be reduced in the prodrug form of the compound, and can be activated by conversion of the prodrug to release the compound or a metabolite of it.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Pharmaceutical Compositions

GPI-PLD proteins may be administered alone or in combination with other treatments for pre-eclampsia. GPI-PLD proteins and any accompanying compositions can be formulated in pharmaceutical compositions, which may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilisers, buffers, antioxidants and/or other additives may be included as required.

The compounds of used according to the present invention are preferably given to an individual in a “prophylactically effective amount” or a “therapeutically effective amount” (as the case may be, although prophylaxis may be considered therapy), this being sufficient to show benefit to the individual, e.g. the amelioration of one of more symptoms of pre-eclampsia, e.g. a reduction in platelet aggregation, a reduction in thrombus formation or a reduction of glomerulonephritis. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition may be administered alone or in combination with other treatments, either simultaneously or sequentially dependent upon the condition to be treated. By way of example, the treatments disclosed in this application may be combined with the use of P-type IPG antagonists disclosed for the treatment of pre-eclampsia in WO98/10791. An example of one class of IPG-P antagonists are antibodies, e.g. the polyclonal and monoclonal antibodies described in WO98/11117 and WO99/47565.

In a further aspect, the present invention provides an article of manufacture containing materials useful in the diagnosis and/or treatment of pre-eclampsia. The article of manufacture comprises a container and a label. Suitable containers include bottles, vials, syringes and test tubes, for example formed from materials such as glass or plastic. The container holds a composition which is effective for diagnosing or treating pre-eclampsia and may have a sterile access port, e.g. a stopper which can be pierced by a needle to allow access to the composition. Where the article is used for the prevention or treatment of pre-eclampsia, the composition may comprises GPI-PLD, or a variant, active portion or fragment thereof. The article may optionally comprise one or more further active ingredients, in admixture with the GPI-PLD or as part of a kit in one or more further containers, for the treatment of pre-eclampsia or to improve the pharmacological properties of the GPI-PLD. Where the article is used for the diagnosis of pre-eclampsia, the container may be used to hold one or more reagents useful in carrying out the assays described herein. The label on or associated with the container indicates the use of the composition for the diagnosis or treatment of pre-eclampsia and may provide detailed instructions to direct the user. The article for manufacture may further comprise a second container comprising a pharmaceutically acceptable buffer, Ringer's solution and/or dextrose solution.

Diagnostic Assays

Methods for determining GPI-PLD activity or the amount or concentration of this enzyme in biological samples from individuals are well known in the art and can be employed in the context of the present invention to determine whether an individual has or is at risk of developing pre-eclampsia or one of the other conditions discussed herein. The purpose of such analysis may be used for diagnosis or prognosis to assist a physician in determining the severity or likely course of the pre-eclampsia and/or to optimise treatment of it.

Preferred diagnostic methods rely on the determination of GPI-PLD activity as abnormal or dysregulated GPI-PLD activity present in the placenta is believed to be one of the causes of pre-eclampsia. In one embodiment, this method comprises determining GPI-PLD activity in a biological sample obtained from the patient, e.g. by measuring the action of GPI-PLD on a labelled substrate. Alternatively or additionally, the concentration or amount of GPI-PLD in a sample may be determined. Typically, the methods employ biological samples such as blood, serum, tissue samples (especially placenta) or urine. Depending on the sample, it may be advantageous to carry out a pretreatment step, e.g. to remove cellular debris or unwanted contaminants from the sample.

In some embodiments, the assay methods for determining GPI-PLD activity employ a binding agent having binding sites capable of specifically binding to GPI-PLD in preference to other molecules, and especially other molecules likely to be present in the sample. Examples of binding agents include antibodies, receptors and other molecules capable of specifically binding GPI-PLD. Conveniently, the binding agent is immobilised on solid support, e.g. at a defined location, to make it easy to manipulate during the assay. This can be achieved using techniques well known in the art such as physisorption or chemisorption, e.g. employing biotin/avidin or biotin/streptavidin to chemically link the binding agent to the solid support.

The sample is generally contacted with a binding agent under appropriate conditions so that GPI-PLD present in the sample can bind to the binding agent. GPI-PLD activity can be determined by employing a suitable substrate, such as glycosylphosphatidylinositol which has a phosphodiester bond cleaved by GPI-PLD to release phosphatidic acid and a GPI-anchor, and monitoring the enzymatic reaction, e.g. by following the amount of substrate or reaction product(s), in the assay system. This process can be facilitated by labelling the substrate, and detecting the reduction in the label on the substrate or the increase of the label in a reaction product.

In the case of determinations of the amount of GPI-PLD in the sample (rather than its activity), the fractional occupancy of the binding sites of the binding agent can then be determined using a developing agent or agents. The developing agent can be used in a competitive method in which the developing agent competes with the analyte for occupied binding sites of the binding agent (e.g. using a labelled analogue of the analyte), or non-competitive method, in which the labelled developing agent binds analyte bound by the binding agent or to occupied binding sites (e.g. using an antibody with appropriate binding specificity). Both methods provide an indication of the number of the binding sites occupied by the analyte, and hence the concentration of the analyte in the sample, e.g. by comparison with standards obtained using samples containing known concentrations of the analyte.

Typically, the substrates or developing agents are tagged with a label or reporter molecule which are directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. Any method known in the art for separately conjugating the antibody to the detectable moiety may be employed, including those methods described by Hunter et al, Nature 144:945, 1962; David et al, Biochemistry 13:1014, 1974; Pain et al, J. Immunol. Meth. 40:219, 1981; and Nygren, J Histochem. and Cytochem. 30:407, 1982.

One favoured mode is to covalently link of each species of substrate or developing agent with an individual fluorochrome, phosphor or laser dye with spectrally isolated absorption or emission characteristics. Suitable fluorochromes include fluorescein, rhodamine, luciferin, phycoerythrin and Texas Red. Suitable chromogenic dyes include diaminobenzidine. Other detectable labels include radioactive isotopic labels, such as ³H, ¹⁴C, ³²P, ³⁵S, ¹²⁶I, or ^{99m}Tc, and enzyme labels such as alkaline phosphatase, β-galactosidase or horseradish peroxidase, which catalyze reactions leading to detectable reaction products and can provide amplification of signal.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyze reactions that develop or change colours or cause changes in electrical properties, for example. They may be molecularly excitable, such that electronic transitions between energy states result in characteristic spectral absorptions or emissions. They may include chemical entities used in conjunction with biosensors.

As mentioned above, the substrates or developing agents are labelled (e.g. with radioactive, fluorescent or enzyme labels) so that they can be detected using techniques well known in the art. Thus, radioactive labels can be detected using a scintillation counter or other radiation counting device, fluorescent labels using a laser and confocal microscope, and enzyme labels by the action of an enzyme label on a substrate, typically to produce a colour change.

Experimental Description

The present invention is based on the realisation that GPI-PLD enzymatic activity is present in the placenta, but the protein responsible for this activity may not itself be expressed in the placenta. GPI-PLD is known to circulate in the blood, complexed with apolipoprotein A1 (ApoA1), and so the placental GPI-PLD may be derived from the maternal circulation.

The foetus is genetically different from the mother, because half the foetus's genes are inherited from the father. The placenta is foetal in origin, rather than maternal. This raises the possibility that paternally-encoded factors in the placenta may be involved in uptake or processing of maternally-derived GPI-PLD, or may affect its activity in situ. Thus, paternal factors may have a bearing on GPI-PLD activity in the placenta. Consequently, without wishing to be bound by any particular theory, an incompatibility between the maternal GPI-PLD and paternally encoded factors in the placenta may result in abnormal GPI-PLD activity in the placenta, causing the unusually high level of P-type IPGs seen in the placenta and urine, as well as the dysregulated carbohydrate metabolism observed in pre-eclampsia. Thus administration of exogenous GPI-PLD may be therapeutically effective in prevention or treatment of pre-eclampsia. Such administration may be in conjunction with ApoA1, to provide an alternative circulating source of GPI-PLD. However, in a particularly preferred embodiment GPI-PLD is administered without ApoA1. In this case it is thought that the exogenous GPI-PLD may compete with the endogenous maternal GPI-PLD for binding sites on maternal ApoA1. Consequently, the exogenous GPI-PLD may displace the endogenous enzyme, reducing the amount of endogenous GPI-PLD reaching the placenta, and replacing it with the exogenous enzyme. In a further embodiment, the present invention provides the use of a GPI-PLD antagonist, e.g. an enzymatically inactive form of the enzyme which can compete with abnormal or dysregulated GPI-PLD causing pre-eclampsia and/or displace such GPI-PLD, e.g. from Apo-A1.

### EXAMPLE 1

### GPI-PLD is Present in the Placenta

Extracts of placenta were prepared and tested for the presence of GPI-PLD by immunoblotting with a polyclonal antiserum directed against the serum form of GPI-PLD. Whole placenta (lane 1) or placental villous tissue (lanes 2-6) was homogenised in buffer (0.1M NaCl, 0.01M Tris HCl pH 7.6, 0.001M EDTA, 1 μg/ml aprotinin, 100 μg/ml phemylmethylsulfonyl fluoride). 0.3 mg of protein was denatured by boiling in gel loading buffer (0.125M tris pH 6.8 containing 4% SDS, 20% glycerol, 10% mercaptoethanol, 0.004% bromophenol blue) and separated by SDS-PAGE on an 8% acrylamide resolving gel. Proteins were then transferred by electroblotting to a nitrocellulose membrane. The GPI-PLD protein was visualised by antibody staining, using the ECL+ Western blot detection system (Amersham) and following the manufacturer's protocol. The polyclonal anti-GPI-PLD antiserum was obtained from La Roche.

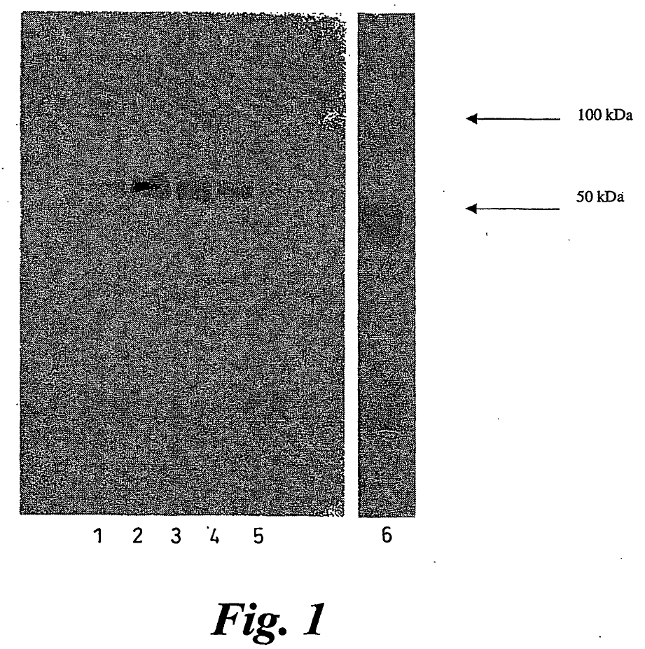

FIG. 1 shows a Western blot showing the presence of GPI-PLD in homogenate of placenta (lane 1) and microvilli preparations of normal (lanes 2, 3, 4, 6) and pre-eclamptic (lane 5) placenta. The polyclonal anti-GPI-PLD antibody recognises a protein of approximately 50 kDa molecular weight, which is believed to represent the product of intracellular cleavage of the GPI-PLD protein taken up from maternal serum. The 100 kDa serum-form of the enzyme can however be detected in some samples, although it is not the predominant form of the enzyme.

### EXAMPLE 2

### Presence of Phospholipase D Enzyme Activity in the Placenta

The presence of phospholipase enzymatic activity in microvilli was investigated using a GPI-anchor hydrolysis assay.

Extracted microvillous membrane was solubilised in buffer A (150 mM NaCl, 10 mM HEPES pH 7.0, 0.1% NaN₃, 0.5% Nonidet P40) and incubated at 37° C. with an equal volume of substrate reaction (40 mM TrisMaleate pH 7.0 containing 0.1% Nonidet P40 and 6 μg of [³H]Myristate-labelled variant surface glycoprotein ([³H]mfVSG), which had been previously prepared. The reaction was stopped by addition of 500 μl 1M NH₄OH-saturated butanol and phase separation achieved by centrifugation (13000 rpm, 5 minutes). ³⁰⁰ μl of the upper organic phase was then scintillation counted.

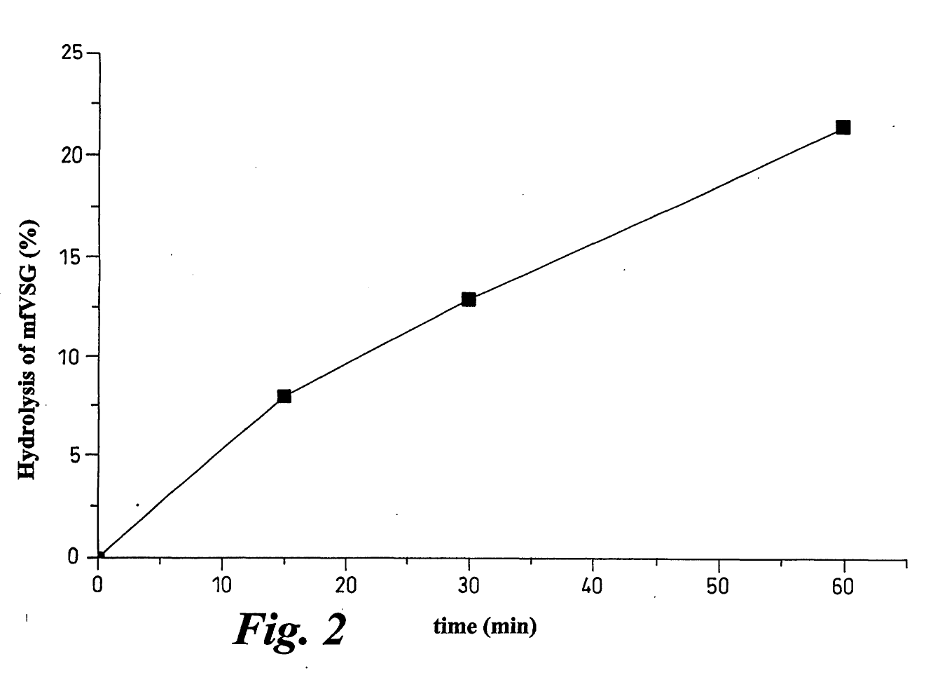

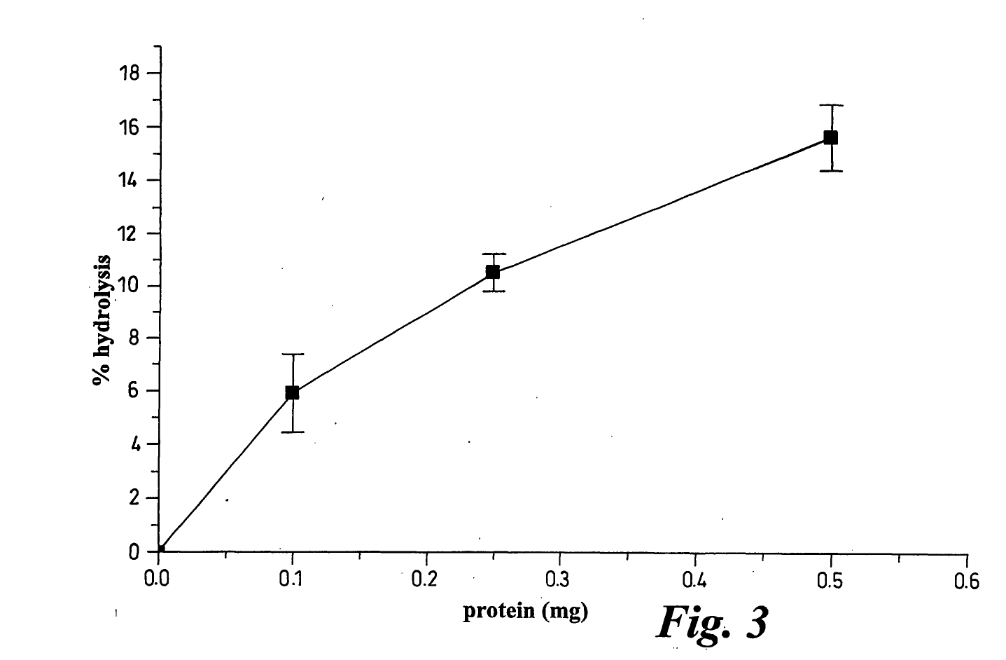

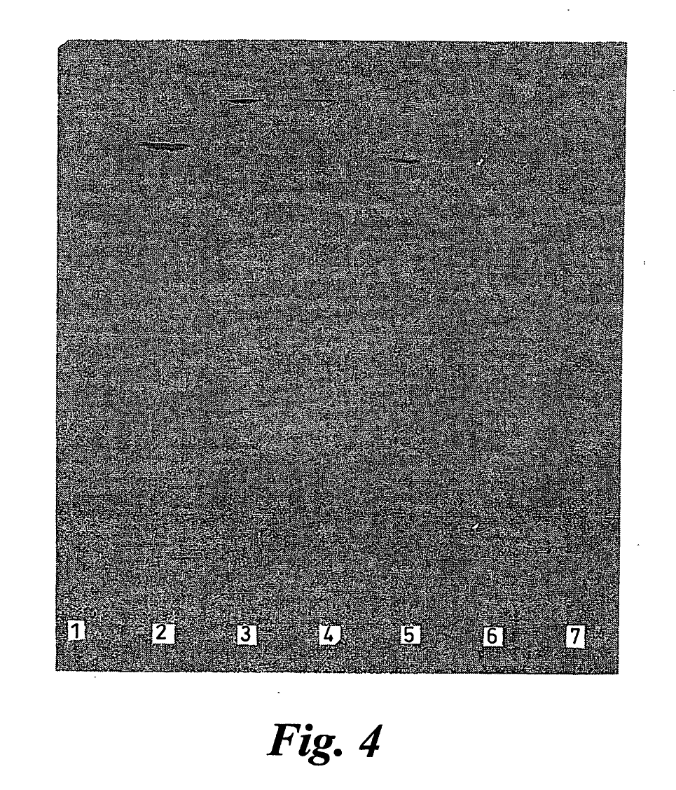

FIGS. 2 and 3 confirm the presence of a GPI-specific phospholipase activity in placental microvilli using hydrolysis of mfVSG. The time of incubation (FIG. 2) or protein concentration (FIG. 3) were varied, and both showed a positive correlation with % mfVSG hydrolysis.

As shown in FIG. 2, this resulted in time-dependent hydrolysis of the mfVSG, with the radiolabel moving into the organic phase and the soluble form of the protein (sVSG) being liberated in the aqueous phase. In FIG. 3, it is shown that the rate of hydrolysis of mfVSG is dependent upon the amount of microvillus tissue present in the assay, proving that the hydrolysis shown in FIG. 2 is due to factors present in the placenta, rather than spontaneous hydrolysis of the GPI-anchor.

### EXAMPLE 3

### Class of Phospholipase Activity

In order to determine the class of phospholipase activity responsible for the mfVSG hydrolysis, the assay was repeated and the hydrolysis products analysed by thin layer chromatography. Hydrolysis of GPI anchors by phospholipase C is known to yield diacyl glycerol. By contrast, phospholipase D activity would be expected to generate phosphatidic acid.

0.25 mg of placental microvillous membrane protein extracted from normal (lanes 4,5) or pre-eclamptic (lanes 6,7) placenta was incubated with [³H]mfVSG for 3 hours at 37° C. in the absence (lanes 4,5) or presence (lanes 5,7) of phosphatase inhibitors 50 mM sodium fluoride and 2.6 mM sodium orthovanadate. As controls, the same amount of substrate was also incubated with either purified bovine GPI-PLD (lane 2) of Trypanosoma brucei GPI-PLC (lane 3).

After stopping the reaction with NH₄OH-saturated butanol, 300 μl of the organic phase was dried using a Speed-Vac concentrator and then resuspended in 20 μl chloroform/methanol (2:1 v/v). The resuspended lipid products were then spotted onto a TLC plate and developed in chloroform/methanol/0.25% KCl (55:45:10). After air drying, the plate was sprayed with En3Hance and exposed to autoradiographic film for 2 weeks.

The phosphatidic acid product of GPI-PLD cleavage (lane 2) is seen in microvilli from both normal (lane 5) and pre-eclamptic placenta (lane 7) when phosphatase inhibitors are included in the reaction.

FIG. 4 shows an autoradiograph of a TLC plate showing that the GPI-specific phospholipase activity present in placental microvilli is of the phospholipase D type. The reaction products are shown in FIG. 4.

Lane 1 mfVSG incubated in reaction buffer alone;

Lane 2 phosphatidic acid;

Lane 3 diacyl glycerol;

Lane 4 mfVSG+normal placental microvilli;

Lane 5 mfVSG+normal placental microvilli+phosphatase inhibitors;

Lane 6 mfVSG+pre-eclamptic placental microvilli;

Lane 7 mfVSG+pre-eclamptic placental microvilli+phosphatase inhibitors.

Incubation of mfVSG with extracts of both normal and pre-eclamptic microvilli yields products with the mobility of both diacyl glycerol and phosphatidic acid. Addition of the phosphatase inhibitors sodium fluoride and sodium orthovanadate to the reaction mixture results in the disappearance of the diacyl glycerol band, along with concomitant increase in intensity of the phosphatidic acid band.

Thus, the immediate product of the hydrolysis reaction is phosphatidic acid, which is then dephosphorylated to diacyl glycerol by contaminating phosphatases. This data therefore clearly demonstrates the presence of a phospholipase D activity in placental microvilli.

### EXAMPLE 4

### Phospholipase D is Not Expressed in the Placenta

mRNA was prepared from samples of both normal and pre-eclamptic placentae, and cDNA prepared by reverse transcription from each sample. 100 mg of tissue from placentae, frozen in liquid nitrogen soon after delivery, was homogenised in RNAzolB reagent (Biogenesis Ltd., Poole, UK) according to the manufacturer's instructions. 5 μg of total RNA extracted in this manner was reverse transcribed into cDNA using Superscript II (Gibco BRL) in a reaction volume of 35 μl primed with random hexamers (Pharmacia). 2.5 μl of this reaction was then amplified by PCR in a reaction volume of 50 μl in the presence of 50 pmoles of 25mer oligonucleotides specific for the human GPI-PLD gene (forward oligo: TTCTTGGAGGACTGGATGATATGGC, reverse oligo: TGAGAGCCACCTATGAACATTGTCC) or cytoplasmic β-actin gene (forward oligo: ATGGATGATGATATATCGCCGC, reverse oligo: ATCTTCTCGCGGTTGGCCTT). PCR of 30 denaturing (94° C., 25 secs), annealing (GPI-PLD: 63° C.,: β-actin: 52.3° C., 30 secs) and extension (72° C., 30 secs) cycles were performed using Taq polymerase (Promega).

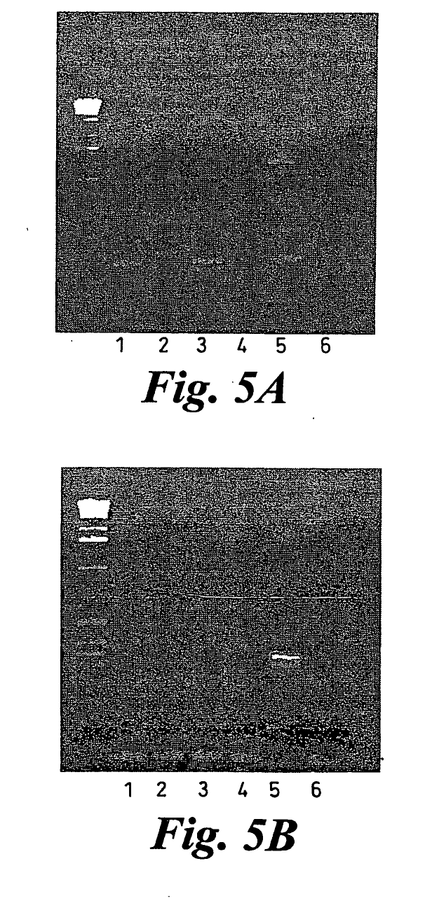

FIG. 5 shows RT-PCR reactions showing that the gene encoding GPI-PLD is not expressed in either normal or pre-eclamptic placentae.

Lanes 1 and 3 contain cDNA made from normal and pre-eclamptic samples respectively. Lanes 2 and 4 are controls (for normal and pre-eclamptic samples respectively) from which the reverse transcriptase enzyme was omitted. Any products seen in these samples must therefore result from amplification of contaminating genomic DNA. Lane 5 contains positive controls; for B-actin, a universal human cDNA library, and for GPI-PLD, a cDNA clone isolated from a human liver cDNA library. Lane 6 contains no cDNA and therefore serves as a negative control for the PCR. The presence of product of expected size (353 bp) in the β-actin PCR with cDNA made from both normal and pre-eclamptic placenta, shows that the RNA synthesis was successful. The larger product of 488 bp is only obtained from genomic DNA. However, the expected GPI-PLD reaction product of 313 bp is not seen in either sample. Since the GPI-PLD PCR was successful (lane 5), it can therefore be concluded that the gene encoding GPI-PLD (GPLD1) is not expressed in the placenta.

Thus, no GPI-PLD mRNA can be detected in human term placenta, despite the demonstrable presence of GPI-PLD enzymatic activity in this tissue. This leads to the conclusion that the enzyme in microvilli is not expressed in the placenta but is derived from the maternal reservoir of circulating enzyme. The microvilli form the barrier layer between mother and foetus which mediates uptake of nutrients from the maternal to the foetal circulation. Thus molecules taken up from the maternal circulation would be expected to be detectable in the microvilli. These findings are therefore entirely consistent with GPI-PLD being taken up by the placenta from the maternal circulation.

### REFERENCES

The references cited herein are all expressly incorporated by reference in their entirety.

Arkwright et al, J. Clin. Invest. 91: 2744-2753, 1993.

Ausubel et al, Short Protocols in Molecular Biology, John Wiley and Sons, 1992

Caro et al, Biochem. Molec. Med., 61:214-228, 1997.

Heller et al, Eur. J. Biochem., 224:823-833, 1994.

Hoener et al, FEBS Lett., 327: 203-206, 1993.

Huang et al, U.S. Pat. No. 5,418,147.

Rademacher et al, Brazilian J. Med. Biol. Res., 27:327-341, 1994.

Redman, Placenta, 12: 301-308, 1991.

Roberts et al, Am. J. Obstet. Gynecol. 161: 1200-1204, 1989.

Robertson et al, J. Path. Bacteriol. 93; 581-592, 1967.

Sambrook, Fritsch and Maniatis, Molecular Cloning, A Laboratory Manual, Cold Spring Harbor Laboratory Press, 1989

Thompson et al, Nucleic Acid Research, 22:4673-4680, 1994, with algorithm from Higgins et al, CABIOS, 8(2):189-191, 1992.

Tsang et al, FASEB J. (supp), 6:A1922, 1992.

1. Use of an antagonist of endogenous glycosylphosphatidylinositol phospholipase D (GPI-PLD) for the preparation of a medicament for the prevention or treatment of pre-eclampsia.

2. The use of claim 1, wherein the GPI-PLD antagonist competes with or displaces endogenous GPI-PLD which causes pre-eclampsia.

3. The use of claim 2, wherein the GPI-PLD antagonist displaces endogenous GPI-PLD from apolipoprotein A1.

4. The use of claim 2, wherein the GPI-PLD or antagonist competes with endogenous GPI-PLD in placenta.

5. The use of any one of the preceding claims, wherein the antagonist is exogenously administered GPI-PLD.

6. The use of claim 5, wherein the GPI-PLD is inactive or has a reduced activity.

7. The use of claim 6, wherein the activity is cleavage of a phosphodiester bond of a glycosylphosphatidylinositol.

8. The use of claim 7 wherein the cleavage of the phosphodiester bind releases P-type inositol phosphoglycans (IPGs).

9. The use of any one of the preceding claims, wherein the GPI-PLD is a fragment of full length GPI-PLD having the amino acid sequence as set out in FIG. 6.

10. The use of any one of the preceding claims, wherein the GPI-PLD is produced by a host cell capable of expressing and secreting GPI-PLD.

11. The use of claim 10, wherein the host cell is encapsulated in a biocompatible polymer, so that the GPI-PLD produced by the host cell can be secreted into the patient, while preventing rejection of the host cell by the immune system of the patient.

12. A method of diagnosing a patient who has or at risk of developing a pre-eclampsia, the method comprising determining the amount of GPI-PLD and/or GPI-PLD activity in a sample obtained from the patient.

13. The method of claim 12, wherein the method comprising the steps of:
(a) contacting a sample obtained from the patient with a solid support having immobilised thereon a binding agent having binding sites specific for GPI-PLD;
(b) determining the amount of GPI-PLD or the activity of GPI-PLD which binds to the binding agent.

14. The method of claim 13, wherein the method comprises the additional step of:
(c) correlating the value obtained in step. (b) with measurements obtained from control subjects to determine whether the patient has or is at risk of developing the condition.
